(19) United States
(12) Patent Application Publication
(10) Pub. No.: US 2008/0114163 A1
(43) Pub. Date: May 15, 2008

(54) Carbonylated Compounds Preparation Process From A Vegetable Product And Resulting Carbonylated Compounds

(76) Inventors: Renato Augusto Konrath, Novo Hamburgo/RS (BR); Francisco Jose Fava, Porto Alegre/RS (BR)

Correspondence Address: OSTROLENK FABER GERB & SOFFEN, 1180 AVENUE OF THE AMERICAS, NEW YORK, NY, 100368403, US

(21) Appl. No.: 11/813,350

(22) PCT Filed: Feb. 13, 2006
(86) PCT No.: PCT/BR06/00023

§ 371 Date: Jul. 5, 2007

(30) Foreign Application Priority Data
Feb. 11, 2005 (BR) PI 0500470-5

(51) Int. Cl. C07H 1/00

(52) U.S. Cl. 536/124

(57) ABSTRACT

The present invention refers to a carbonylated compounds preparation process from a vegetable product and resulting carbonylated compounds. The vegetable product mentioned is from renewable resources. These carbonylated compounds can be used replacing several products mainly aldehydes.

The process proposed comprises the carbohydrate activation that consists on getting the carbohydrate in touch with the Lewis' acid in order to facilitate a following nucleophilic substitution process. This activation occurs at same temperature of the carbohydrate preparation process.

The mixture nucleophile/carbohydrate at controlled conditions will generate substituted carbonylated compounds. This process is carried on temperatures not lower than 20° C. and not higher than 105° C., for running time not lower than 10 minutes.

It is necessary to stabilize the resulting substituted aldehyde mixture with any suitable stabilizing agent for this purpose. Stabilizing agent proportion will vary from 0 to 5% in mass. A biocide is added in order to eliminate the possibility of bacterial degradation of any remaining non-reacted carbohydrates residual. This biocide is added in proportions that vary from 0 to 1% in mass.

The present invention describes a carbonylated compounds preparation process from a vegetable product and a resulting carbonylated compounds. The vegetable product mentioned is from renewable resources. These carbonylated compounds replace several products currently used like aldehydes.

At present carbonylated compounds can be obtained in several ways in the industry. Most commonly is the primary alcohol oxidation that generates aldehydes and secondary alcohol, alkenes or arenes oxidation that produces ketones. As aldehydes are intermediates compounds between alcohols and ketones its oxidation should be controlled carefully.

Apart from oxidation processes, there are many other ways to produce carbonylated compounds. For producing aldehydes, for example, an acyl chloride, esters or nitrile reduction with aluminum or lithium hydrate or similar compounds can be done. For producing ketones a lithium dialkylcuprates and acyl chlorides reaction can be used. There are other less common methods that can also be used.

Carbonylated compounds have many industrial uses as raw material for chemical industry, for resin and polymer production, odoriferous substances for perfume industry, chemical reagents for synthesis and analysis and many other purposes.

This invention relates to new a compounds synthesis method through acid hydrolysis of carbohydrates in order to produce aldehydes and ketones mixture of varied molecular weights, according to carbohydrates source used. Acid hydrolysis is carried out on presence of a typical Lewis' acid to replace produced carbonylated compounds (normally hidroxycarbonylated) by a nucleophile. At this moment a nucleophilic substitution refrains an eventual undesirable formation of cyclic carbonylated compounds and stabilize formed products as a shield against bacterial attack.

The related product of this invention is prepared as a chemical mixture of the following:
a) a carbohydrate or a mixture of carbohydrate
b) a nucleophile
c) a Lewis' acid
d) a stabilizing agent
e) a biocide

So this invention refers to a new carbonylated compound preparation method comprising the following steps:
a) carbohydrate preparation
b) carbohydrate activation with a Lewis' acid
c) mixture of activated carbohydrate to a nucleophile
d) stabilizing agent addition to biocide

Carbohydrates employed are of a natural source and can be represented by any of the following group, but not limited to: monosacharydes, dissacharides, polysaccharides and gums. (exemple: frutose, glucose, gulose, arabinose, manose, ribose, sucrose, lactose, celobiose, starch, guar gum, acácia gum, xanthum gum, tragacanth gum, arabic gum, alginates, etc.)

Lewis' acids can be represented, but not limited to the following: aluminum chloride, zinc chloride, ferric chloride, boron trifluoride, ferric bromide, boron trifluoride, magnesium bromide, magnesium chloride, zinc bromide, suitable nucleophile agent used can be one, but not limited to, the following compounds: bromides, chlorides, iodide, fluorides, ammonia, thiophenoxide, phenoxydes, alcoxides, sulfoxides, etc. . . .

Carbohydrate preparation consists in submitting carbohydrate to an acid or alkaline hydrolysis in order to break up glucose chemical bonds to form saccharides of lower molecular weight. This process is carried on at temperature ranging 25 to 105° C. and times reactions ranging from 15 minutes to 48 hours according to polysaccharide chosen for the process.

Carbohydrate activation consists on getting the carbohydrate in touch with the Lewis' acid in order to facilitate a following nucleophilic substitution process. This activation occurs at same temperature of the carbohydrate preparation process.

The mixture nucleophile/carbohydrate at controlled conditions will generate substituted carbonylated compounds. This process is carried on temperatures not lower than 20° C. and not higher than 105° C., for running time not lower than 10 minutes.

It is necessary to stabilize the resulting substituted aldehyde mixture with any suitable stabilizing agent for this purpose. Stabilizing agent proportion will vary from 0 to 5% in mass. Sodium metabissulfite is an example of stabilizing agent that will produce addition compounds with aldehydes and will avoid the typical condensation reaction of its carbonyl group, common in this class of compounds.

A biocide addition will eliminate the possibility of bacterial degradation of any remaining non-reacted carbohydrates residual. This biocide is added in proportions that vary from 0 to 1% in mass.

A colorless to yellowish syrup like liquid is obtained that can be used in the purposes mentioned before.

The present invention will be better understood in the following examples but not limited to:

### EXAMPLE 1

To a closed 5 liter capacity reactor with a stirring and heating/cooling device we add 2 kg starch, 2.8 kg of water and 100 grams of hydrochloric acid. Keep stirring for 12 hours at 50° C. temperature. After this time a yellowish liquid is obtained to which is added more 72 grams of aluminum trichloride and 92 grams of hydrochloric acid. Stir it further 5 hours at 40° C. in order to obtain a more clear liquid. To this liquid it is added more 300 g of sodium bissulphite as a stabilizing agent and 20 grams of a biocide and stir 1 hour further at the same temperature. Afterwards pH value is checked and raised to 6 by adding a sodium hydroxide solution. This product is a yellowish syrup like liquid and can be used as described previously.

### EXAMPLE 2

To a closed 5 liter capacity reactor with a stirring and heating/cooling device we add 18 kg of glucose, 2.7 kg of water, 68 gram aluminum trichloride and 170 g of hydrochloric acid. Stir for 2 hours at a temperature of 60° C. A clear liquid is obtained to what is added 250 gram sodium bissulphite as a stabilizing agent and 18 grams of biocide. Stir it for more one hour at the same temperature. Afterwards the pH value of this mixture is checked and raised by adding a sodium hydroxide solution. This product is a yellowish syrup like liquid and can be used as described previously.

1- “CARBONYLATED COMPOUNDS PREPARATION PROCESS FROM A VEGETABLE PRODUCT AND RESULTING CARBONYLATED COMPOUNDS”, is characterized by the fact of comprising three steps:
a) Carbohydrated preparation that consists in submitting carbohydrate to an acid or alkaline hydrolysis in order to form saccharides of lower molecular weight at temperature ranging 25 to 105° C. and time reactions ranging from 15 minutes to 48 hours.
b) Carbohydrate activation consists on getting the carbohydrate a) in touch with a Lewis' acid at same temperature range of the carbohydrate preparation process.
c) Mixture of prepared and activated in a) and b) above mentioned with a nucleophile agent at temperatures between 20° C. and 105° C., for running time not lower than 10 minutes in order to generate a mixture of substituted carbonylated compounds
d) Stabilization of substituted carbonylated compounds obtained in c) above by means of an adequate stabilizing agent in proportion that will vary from 0 to 5% in mass. In this step any alkali can be added to raise the pH value to the range of 5 to 6.
e) The addition of a biocide to the mixture obtained in d) to prevent the possibility of bacterial degradation of any remaining non-reacted carbohydrates residual. A colorless to yellowish syrup like liquid is obtained.

2- “CARBONYLATED COMPOUNDS PREPARATION PROCESS FROM A VEGETABLE PRODUCT AND RESULTING CARBONYLATED COMPOUNDS”, is characterized by the fact that, according to claim 1, the obtained products can be used as a mixture of carbonylated compounds (aldehydes, ketenes, etc.) in several processes where aldehydes or ketones are used.
